(19) United States
(12) Patent Application Publication
(10) Pub. No.: US 2024/0225984 A9
(43) Pub. Date: Jul. 11, 2024

(54) TOPICAL COMPOSITION FOR REGULATION OF INFLAMMATION AND SKIN REGENERATION

(71) Applicant: Glycopep Therapeutics, S.A.S. de C.V., Aguascalientes (MX)

(72) Inventors: Oscar Antonio ORTEGA RIVERA, Aguascalientes (MX); Oscar Ivan RAMIREZ LOPEZ, Aguascalientes (MX)

(73) Assignee: Glycopep Therapeutics, S.A.S. de C.V., Aguascalientes (MX)

(21) Appl. No.: 18/490,806

(22) Filed: Oct. 20, 2023

(30) Foreign Application Priority Data
Oct. 24, 2022 (MX) MX/A/2022/013371

(51) Int. Cl. A61K 8/64; A61K 8/67; A61K 31/164; A61K 47/54; A61P 29/00; A61Q 19/00

(52) U.S. Cl. CPC A61K 8/64; A61K 8/673; A61K 31/164; A61K 47/549; A61P 29/00; A61Q 19/00

(57) ABSTRACT

A topical composition useful in the treatment of inflammatory skin diseases, including glycomacropeptide (GMP), panthenol, and a cosmetologically acceptable vehicle.

### FIELD OF THE INVENTION

The present invention belongs to the biotechnology area, specifically to the use of macroglycopeptides in the treatment of inflammation and skin regeneration.

### BACKGROUND OF THE INVENTION

The skin is the organ that covers the surface of the body, it is mainly made up of keratinocytes which are responsible for regulating external and internal stimuli and responses. Constant interactions on the epidermal barrier generate a loss of homeostasis, triggering multiple aberrant responses of keratinocytes that eventually manifest in alterations of the epidermal barrier, generating itching, pain, wounds, the loss of its integrity and the deregulation of its immunological components. (Gallegos-Alcala P, Jimenez M, Cervantes-Garcia D, Salinas E. The Keratinocyte as a Crucial Cell in the Predisposition, Onset, Progression, Therapy and Study of the Atopic Dermatitis. Int J Mol Sci. 2021 Oct. 1; 22(19):10661. doi: 10.3390/ijms221910661. PMID: 34639001; PMCID: PMC8509070.).

These are considered causes and/or symptoms of inflammation, and therefore are also relevant objectives to treat to reduce the damage caused by these conditions in the short, medium and long term. (Hay R. J., Johns N. E., Williams H. C. et al., “The global burden of skin disease in 2010: an analysis of the prevalence and impact of skin conditions,” Journal of Investigative Dermatology, vol. 134, no. 6, pp. 1527-1534, 2014).

Currently, a considerable number of skin diseases have been discovered and described, and the common denominator in all these conditions is immunological inflammation. (Hay et al, 2014). Many of these skin disorders occur at an early age such as childhood and can eventually lead to other inflammatory diseases not related to the skin.

The most common treatments are topical anti-inflammatories whose active ingredients are mainly corticosteroids or calcineurin inhibitors. (Dharmage S. C., Lowe A. J., Matheson M. C., Burgess J. A., Allen K. J., and Abramson M. J., “Atopic dermatitis and the atopic march revisited,” Allergy, vol. 69, no. 1, pp. 17-27, 2014.). However, the relief that these medications provide is temporary and their long-term use is associated with significant adverse effects on the epidermis. (Ference J. D. and Last A. R., “Choosing topical corticosteroids,” American Family Physician, vol. 79, no. 2, pp. 135-140, 2009.) Additionally, these types of inflammatory skin diseases influence people's quality of life, are potentially predecessors of systemic inflammation, in addition to having a high impact on health systems due to being long-term treatments.

On the other hand, generic topical moisturizing formulas with or without active compounds can momentarily favor symptoms related to the loss of the epidermal barrier. However, due to the presence of dyes, fragrances or some other ingredients, these can exacerbate inflammation and the symptoms related to it, so they do not offer a permanent remedy to the skin problem.

In light of the above, there is a need to develop topical compositions for the treatment of skin diseases, without side effects and high effectiveness.

An alternative proposed in the present invention is the use of glycomacropeptide (GMP) together with panthenol and a cosmetologically acceptable vehicle.

GMP is a compound derived from milk proteins, which comprises 64 amino acids and is glycosylated. Generally, this compound is found in whey and is obtained from the hydrolysis of milk when kappa-casein is treated with chymosin. Depending on the original source of the milk, the sequence and composition of GMP is variable both genetically and in polysaccharides. One of these polysaccharides is sialic acid, which is known to have an immunomodulatory effect. GMP has generally been used as a dietary supplement. Among its biological functions is its ability to fight infections, as an anti-caries compound and even as an antihypertensive.

Examples of the uses of GMP are described in the following documents:

Document WO2005037248A1 describes preparations for use in scarring or diseases of superficial mucosa in the eyes, nose, mouth, vagina or rectum. The composition comprises glycomacropeptide derived from whey and is free of lactoferrin, beta-lactoglobulin, alpha-lactalbumin, BSA and further comprises a viscous carrier. This document focuses on eye drops as the preferred modality and does not mention the use of GMP for skin diseases, as is the case with the present invention, nor does it suggest the use of panthenol.

Document ES 2,272,535 T3, describes the use of a casein derivative for the preparation of a composition for the prevention or treatment of pathogen-induced disorders of the cutaneous system, in which the casein derivative is a caseinoglycomacropeptide (CGMP), a glycosylated derivative of caseine-glycomacropeptide or asialic CGMP, wherein the casein derivative is capable of stabilizing and/or regulating the pathogenic flora of the skin system by inhibiting the adhesion of pathogens such as Streptococcus pyogenes, Trichophyton rubrum, Pityrosporum ovale, Candida albicans or M. furfur. However, the use of GMP alone or in combination with panthenol for the treatment of inflammatory skin diseases, as is the case of the present invention, is not described, but is limited to pathogen-induced diseases of the cutaneous system.

Document KR101974405B1 describes a cosmetic composition for skin regeneration, which comprises colostrum hydrolysates (whey) as an active ingredient. Skin regeneration is promoted by increasing the expression of involucrin, cornifin and loricrin related to keratinocyte differentiation and secretion of fibronectin.

The hydrolyzate is prepared by treating the whey protein with an alkaline protease at a pH of between 8 and 10 at a temperature of 50° to 60° C., deactivating the enzyme at a temperature between 80 and 100° C. for between 10 to 40 minutes. However, this document does not involve the isolated use of GMP, but rather colostrum hydrolysates in their entirety as an active ingredient, which is why it widely differs from the scope of the present invention.

Document US20220062161A1 discloses methods to improve skin hydration associated with redness, dryness, improve firmness, youthful appearance, appearance of wrinkles, through oral or topical application of partial whey protein hydrolysate. However, the document details that the composition does not contain GMP, so it constitutes an additional document of the state of the art.

Finally, document MX 365990 describes the use of GMP as an immunomodulator in IgE-mediated allergies, where the composition is administrable orally, intravenously, intramuscularly, intraperitoneally, subcutaneously, transdermally or through mucosa. However, the use of GMP in inflammatory skin conditions or diseases alone or in combination with panthenol and an additional vehicle is not described or suggested.

Thus, considering the above, no document has been found where the use of GMP with panthenol and a cosmetically acceptable vehicle for the preparation of a topical composition useful in the treatment of skin diseases caused by inflammation is described or suggested. The invention will now be described, where the topical composition is described in various modalities as well as its use in 26 skin disorders mediated by the differential expression of markers such as, for example, IL-1B, Cox-2 or TRPA1, GAPDH, HMGB1 or TNF.

### SUMMARY OF THE INVENTION

The present invention describes and claims in a first aspect, a topical composition, characterized in that it comprises glycomacropeptide, sialic acid and panthenol and a cosmetologically acceptable vehicle, wherein the sialic acid is chemically linked to the glycomacropeptide. Preferably, the composition comprises between 0.5 to 10% w/v glycomacropeptide, 0.06-1.2% w/v sialic acid, 0.1-5% w/v panthenol and a cosmetologically acceptable vehicle. Said topical composition is in the form of an emulsion, ointment, lotion, paste, powder, cream, ointment, spray, soap or shampoo, among other forms that allow topical application and also promotes cell regeneration and/or reduces inflammation in keratinocytes. humans.

In a second aspect of the invention, the use of glycomacropeptide, sialic acid and panthenol is described and claimed, to prepare a topical composition for the treatment of conditions mediated by inflammation in the skin, where said conditions are: post-chickenpox, cheilitis, mask dermatitis, superficial burns, cracks, diaper rash, fissures, tattoos, pre-post laser and pulsed light, eczema, severe dryness in feet, elbows and knees, pre-post peeling, pre-post sun exposure, superficial cuts, xerosis, post herpes, small wounds, bites, post-dermabrasion, scrapes, psoriasis, dermatitis due to hygiene measures, constant washing and alcohol, contact dermatitis (jewelry, industry, exposure to chemicals, use of protective equipment, reactions on the skin by pets, acne, miliaria, pruritus, phytotoxicity, friction melanosis or ichthyosis).

A further embodiment of the invention comprises the use of glycomacropeptide, sialic acid and panthenol, to prepare a topical composition to promote cell regeneration in skin.

### BRIEF DESCRIPTION OF THE FIGURES

FIG. 1. Viability of HaCaT cells treated with increasing concentrations of 2.5% GMP, alone or combined with 0.5% and 1% panthenol. Cells were incubated for 1 and 2 h as acute exposure and 24 and 48 h as chronic exposure. Yellow dotted line, viability 50% (LD50). Data were represented as mean—SEM; N=6, 2 tests in triplicate. Black circle, control; Red circle: panthenol. 0.5%; Orange circle: 1% Panthenol. Blue box: GMP at 2.5%; Green box: 2.5% GMP and 0.5% panthenol; Pink box: 2.5% GMP+1% panthenol.

FIG. 2. Viability of HaCaT cells treated with increasing concentrations of 5% GMP, alone or combined with 0.5% and 1% panthenol. Cells were incubated for 1 and 2 h as acute exposure and 24 and 48 h as chronic exposure. Yellow dotted line, viability 50% (LD50). Data were represented as mean—SEM; N=6, 2 tests in triplicate. Black circle, control; Red circle: panthenol. 0.5%; Orange circle: 1% Panthenol. Blue box: GMP at 5%; Green box: 5% GMP and 0.5% panthenol; Pink box: 5% GMP+1% panthenol.

FIG. 3. Effect of the topical composition, increasing cell proliferation. Viability analysis by MTT., one-way ANOVA post hoc bonferroni, *p>0.001, **p>0.0001 vs. Control of each time. 5% GMP blue line, GMP+panthenol green line, 0.5% panthenol yellow line dotted black line, control.

FIG. 4. Measurement of the expression level of IL-1β/GAPDH mRNA (abscissa axis) vs. the application of GMP+panthenol with or without DNCB (ordinate axis). One-way ANOVA post hoc bonferroni+p>0.05, ++p>0.01 vs. Control, *p>0.0001 vs. DNCB 15 μM.

FIG. 5. Measurement of the expression level of Cox-2/GAPDH mRNA (abscissa axis) vs. the application of GMP+panthenol with or without DNCB (ordinate axis). One-way ANOVA post hoc bonferroni+p>0.05, ++p>0.01 vs. Control, *p>0.0001 vs. DNCB 15 μM.

FIG. 6. Measurement of the expression level of TRPA1/CtGAPDH mRNA (abscissa axis) vs. the application of GMP+panthenol with or without DNCB (ordinate axis). One-way ANOVA post hoc bonferroni+p>0.05, ++p>0.01 vs. Control, *p>0.0001 vs. DNCB 15 μM.

### DETAILED DESCRIPTION OF THE INVENTION

The present invention focuses on a topical composition comprising:

Glycomacropeptides (GMP) derived from sweet whey.

Sialic acid of dairy origin

Panthenol

A cosmetically acceptable vehicle, wherein sialic acid is chemically linked to GMP.

The composition is formulated to regulate the immune response in the skin against conditions associated with the loss of homeostasis of keratinocyte responses. These conditions are inflammatory in nature.

The topical composition of the present invention is of natural origin and is purified through a biotechnological process, so it does not contain dyes or fragrances and is free of allergens and corticosteroids, so it does not present the adverse effects associated with said ingredients.

The topical composition of the present invention has characteristics that complement each other and act at two main cellular levels, namely, at the level of keratinocytes (the most abundant cell type in the skin) and at the level of the immune system cells associated with the epidermis. These beneficial effects on inflammatory skin disorders are due to the characteristics of the ingredients in the composition and their synergistic effect on skin cells.

Sialated GMP is a derivative of sweet whey proteins, with a sialic acid content of about 0.6%. It can regulate the immune system, improving the integrity of the epidermal barrier and preventing the colonization of pathogenic bacteria and fungi. Its use has mainly been orally, but the application of whey improves the structure of the epidermal barrier. GMP-associated sialic acid inhibits the adhesion of bacteria to skin cells, helps lighten human (reconstructed) skin and has shown a regulatory effect on the immune system by binding to SIGLEC receptors.

Panthenol or vitamin B5 has a moisturizing effect and helps reestablish the balance of the epidermal barrier, promotes hydration of the stratum corneum, accelerates the regeneration of superficial wounds, reduces redness and roughness of the skin and the characteristic clinical signs of irritant contact dermatitis, and maintains its effects when used short and long term without showing adverse effects.

Cosmetically acceptable vehicles include, but are not limited to: barrier bases (oils, petroleum jelly, waxes, resins, silicones, etc.), emulsions, lotions, liposomes or suspensions, as well as paste, powder, gel, cream, ointment, lotion, tonic, spray, serum, soap, shampoo, conditioners or masks, among others. Thus, the topical composition of the present invention can be in the form of an emulsion, ointment, lotion, paste, powder, cream, ointment, spray, soap or shampoo, among other forms that allow topical application.

In a preferred embodiment, the topical composition of the instant invention comprises:

0.5-10% w/v GMP

0.06-1.2% w/v of sialic acid

0.1-5% w/v of panthenol,

A cosmetically acceptable vehicle

In a specific embodiment, the composition of the invention comprises 5% GMP, 0.6% sialic acid and 5% panthenol and a cosmetologically acceptable vehicle.

The active compounds of the topical composition influence proliferation and as a dermo regulator, which will be demonstrated in the evaluation of the expression of various genetic markers in the examples of the present invention, under acute and chronic exposures to sialidated GMP alone or in combination with panthenol and with or without stimulation with di-nitrochlorobenzene (DNCB) as an inflammation-inducing agent, on human keratinocyte cells.

The topical composition of the present invention is useful in the treatment of inflammation-mediated diseases or conditions in the skin.

Such conditions are, for example: post-chickenpox, cheilitis, mask dermatitis, superficial burns, cracks, diaper rash, fissures, tattoos, pre-post laser and pulsed light, eczema, severe dryness of the feet, elbows and knees, pre- -post peeling, pre-post sun exposure, superficial cuts, xerosis, post herpes, small wounds, bites, post-dermabrasion, scrapes, psoriasis, dermatitis due to hygiene measures, constant washing and alcohol, contact dermatitis (jewelry, industry, exposure to chemicals, use of protective equipment, skin reactions from pets, acne, miliaria, pruritus, phytotoxicity, friction melanosis or ichthyosis).

### EXAMPLES

### Example 1. In Vitro Cell Viability Studies with HaCaT Human Keratinocytes Treated or not with the Topical Composition of the Present Invention

We worked with HaCaT cells, an immortalized human keratinocyte cell line, which were cultured in DMEM medium supplemented (1 g/L glucose, 4 mM L-alanyl-glutamine, 10% inactivated fetal bovine serum, 50 IU/penicillin, 50 μg/ml streptomycin) at 37° C. and 5% CO₂. Cells were worked between passages 3 to 7. They were seeded in 96-well plates, at a density of 10×10³ cells/well and incubated for 24 h at a confluency close to 15%.

They were treated with 2.5 and 5% sialylated GMP, alone or combined with 0.5 and 1% panthenol for 0, 1, 2, 24 and 48 h. To define the percentage of viability of the cells with the different treatments at each of the times tested, cells not treated with either sialidinated GMP or panthenol were used as a control condition.

Once the time was up, cell viability was determined by measuring mitochondrial metabolic activity by spectrophotometric techniques and using tetrazolium salts.

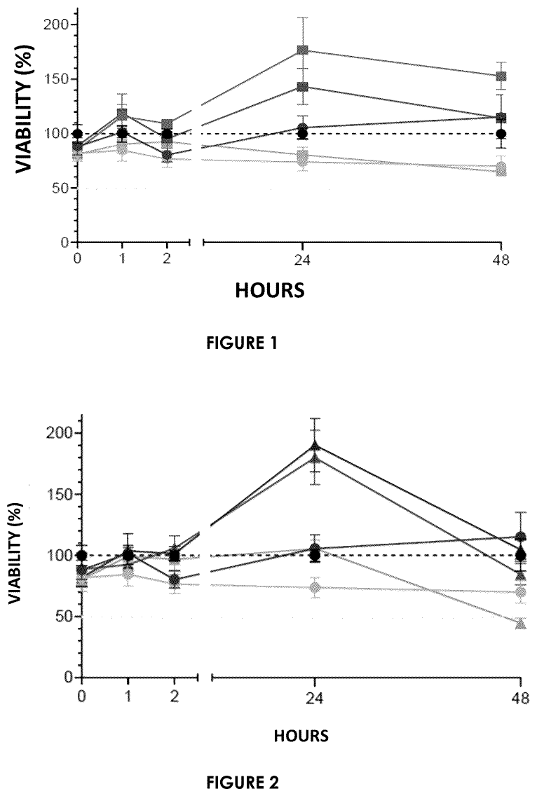

As a result, it was observed that 1% panthenol reduced cell viability by 30% at the different times evaluated, while this effect was only observed when incubating the cells for 2 h with 0.5% panthenol. Furthermore, all treatments with 2.5% GMP, alone or in combination, maintained HaCaT cells with viabilities greater than 50%, both in acute and chronic exposures (FIG. 1). Treatments with 5% GMP, alone or in combination, showed viabilities greater than 50% after 1, 2 and 24 h of incubation. Cells incubated for 48 h with the 5% compound combined with 0.5% panthenol had a viability greater than 50%; However, GMP combined with 1% panthenol generated a viability of less than 50% (FIG. 2). It should be noted that the decrease in viability in the groups containing GMP is not necessarily due to cell death caused by the compound per se, but rather, as observed from hour 24, there is a proliferation of up to 50% greater compared to the initial viability and therefore the space in which the cells can continue proliferating is exceeded in this time (24 h) and eventually viability declines upon reaching 100% confluency.

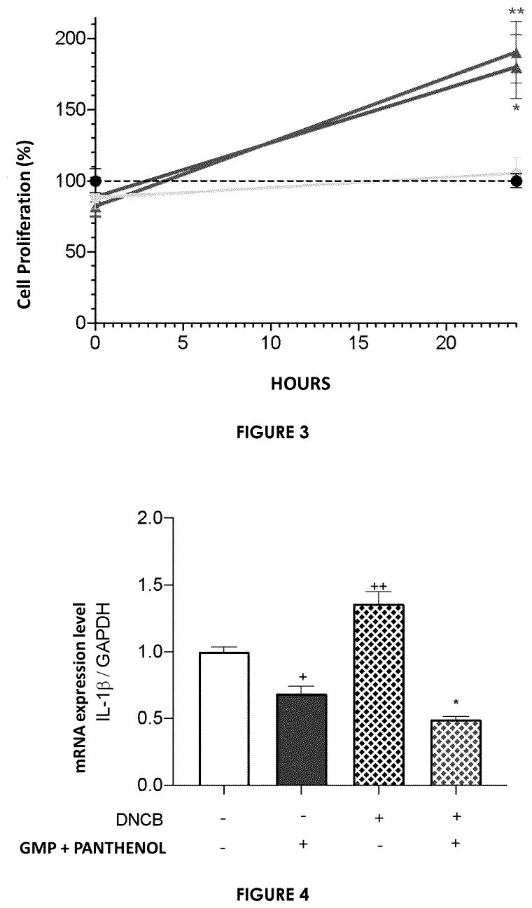

The use of the topical composition of the invention does not show cytotoxicity and promotes cell proliferation in a proportion of 5% sialated GMP and 0.5% Panthenol, as can be seen in FIG. 3.

Thus, the topical composition of the present invention presents a synergistic effect of its components, which allow the regeneration of keratinocytes to be carried out, expressed as cell proliferation in vitro and without cytotoxic effects at the cellular level.

### Example 2. Expression of Cellular Markers Related to Inflammation Using the Topical Composition of the Invention

To verify the action of the topical composition of the present invention on human keratinocytes, tests were carried out with different genetic markers indicative of inflammation and/or skin regeneration.

In vitro studies were carried out to analyze the cell viability of HaCaT human keratinocytes treated or not with the topical composition of the invention.

HaCaT cells, an immortalized human keratinocyte cell line, were cultured in DMEM medium supplemented (1 g/L glucose, 4 mM L-alanyl-glutamine, 10% inactivated fetal bovine serum, 50 IU/penicillin, 50 μg/ml streptomycin) at 37° C. and 5% CO₂. Cells were worked between passages 3 to 7.

They were seeded in 96-well plates, at a density of 10×10³ cells/well and incubated for 24 h at a confluency close to 15%.

They were treated with 2.5 and 5% GMP, alone or combined with 0.5 and 1 panthenol for 0, 1, 2, 24 and 48 h. To define the percentage of viability of the cells with the different treatments at each of the times tested, cells not treated with either GMP or panthenol were used as a control condition.

Once the time was up, cell viability was determined by measuring mitochondrial metabolic activity by spectrophotometric techniques and using tetrazolium salts.

FIG. 3 shows the effect of the topical composition of the present invention, increasing cell proliferation.

To verify that the composition of the present invention regulates the expression of genes associated with inflammation, the expression of IL-1β/GAPDH and Cox-2/GAPDH mRNA was quantified. When keratinocytes are exposed to IL-1β, they increase the levels of messenger RNA of other cytokines such as MIP-2, TNF-α, IL-10 and IL-1α. In this sense, the regulation of IL-1β is beneficial during inflammatory processes in the skin. The regulation of COX-2 is beneficial during inflammatory processes and wound regeneration.

HACaT cells were cultured in supplemented DMEM medium (1 g/L glucose, 4 mM L-alanyl-glutamine, 10% inactivated fetal bovine serum, 50 IU/penicillin, 50 μg/ml streptomycin) at 37° C. and 5% CO₂. Cells were worked between passages 3 to 7.

They were seeded in 6-well plates at a density of 10×10⁵ cells/well and incubated for 24 h at close to 20% confluency. After three days, 75% confluences were achieved.

FIG. 4 shows that the expression level of IL-1β decreased in the presence of GMP alone or in combination with panthenol in relation to control cells. Stimulation with DNCB (di-nitrochlorobenzene as an inflammation-inducing agent) increased the transcription of IL-1β mRNA and treatments with the composition of the invention decreased said expression.

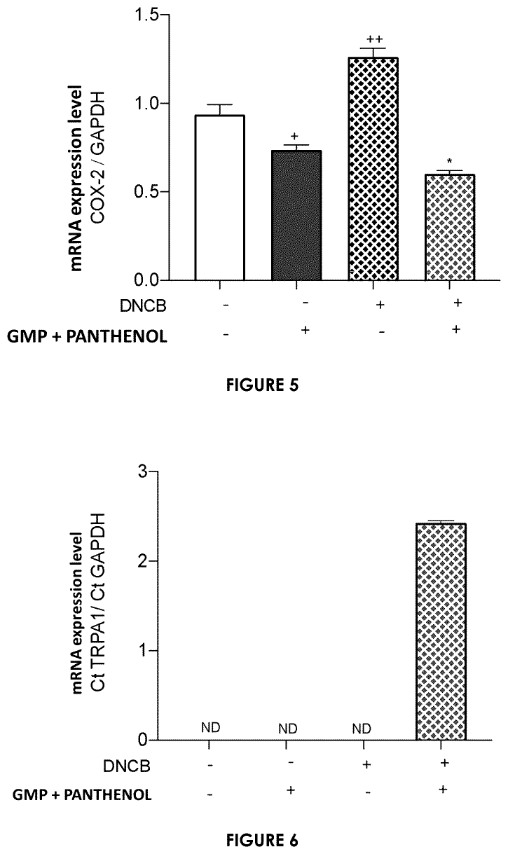

FIG. 5 shows the results obtained for the Cox-2 case in keratinocyte cells. The application of GMP+panthenol reduced the expression of said marker compared to the control. DNCB significantly increased the expression of Cox-2 in these cells, but this increase decreased when GMP+panthenol was applied.

Therefore, the composition of the present invention plays a fundamental role in the regulation of cellular markers associated with inflammation and pain in human keratinocytes.

Now, in relation to skin regeneration, we worked with the marker TRPA1 (transient receptor potential ankryn 1), which serves as a sensor for chemical irritants. Activation of TRPA1 by icilin in keratinocytes leads to an elevation of the proinflammatory cytokine interleukin-1 (IL-1), suggesting a role for TRPA1 in promoting skin inflammation.

Pharmacological inhibition or deficiency of TRPA1 alleviates the inflammation of atopic dermatitis. The mechanism of action related to the use of GMP+Panthenol as an antiprurinergic may be due to a TRPA1 desensitization effect, as can be seen in FIG. 6.

HACaT cells were cultured in supplemented DMEM medium (1 g/L glucose, 4 mM L-alanyl-glutamine, 10% inactivated fetal bovine serum, 50 IU/penicillin, 50 μg/ml streptomycin) at 37° C. and 5% CO₂. Cells were worked between passages 3 to 7.

They were seeded in 6-well plates at a density of 10×10⁵ cells/well and incubated for 24 h at a confluency close to 20%. After three days, 75% confluences were reached.

These cells were treated or not with DNCB and GMP+panthenol. In the case of the simultaneous application of DNCB and the composition of the present invention, an increase in the expression of the marker TRPA1, inducible of skin regeneration, is observed.

1. A topical composition comprising
glycomacropeptide, sialic acid;
panthenol; and
a cosmetically acceptable vehicle.

2. The topical composition according to claim 1, wherein the sialic acid is chemically linked to the glycomacropeptide.

3. The topical composition according to claim 2, wherein said composition comprises 0.5-10% w/v glycomacropeptide, 0.06-1.2% w/v sialic acid, 0.1-5% w/v panthenol and a cosmetically acceptable vehicle.

4. The topical composition according to claim 3, wherein said composition is in the form of an emulsion, ointment, lotion, paste, powder, cream, spray, soap or shampoo among other forms that allow topical application.

5. The topical composition according to claim 1, wherein said composition promotes cellular regeneration.

6. The topical composition according to claim 1, wherein said composition diminishes inflammation in human keratinocites.

7. A method for treating conditions mediated by skin inflammation comprising administering a topical composition comprising glycomacropeptide, sialic acid and panthenol.

8. The method according to claim 7, wherein said conditions are: post-chickenpox, cheilitis, mask dermatitis, superficial burns, cracks, diaper rash, fissures, tattoos, pre-post laser and pulsed light, eczema, severe dryness in feet, elbows and knees, pre-post peeling, pre-post sun exposure, superficial cuts, xerosis, post herpes, small wounds, bites, post-dermabrasion, scrapes, psoriasis, dermatitis due to hygiene measures, constant washing and alcohol, contact dermatitis (jewelry, industry, exposure to chemicals, use of protective equipment, reactions on the skin by pets, acne, miliaria, pruritus, phytotoxicity, friction melanosis or ichthyosis).

9. A method for promoting skin cell regeneration comprising administering to a person in need of a topical composition comprising glycomacropeptide, sialic acid and panthenol.
